(19) United States
(12) Patent Application Publication
(10) Pub. No.: US 2023/0247965 A1
(43) Pub. Date: Aug. 10, 2023

(54) WATER CIRCULATION-TYPE MALODOR REDUCTION SYSTEM

(71) Applicant: TAESUNG ENVIRONMENTAL RESEARCH INSTITUTE CO., LTD., Ulsan (KR)

(72) Inventors: Gi Yeol YUN, Ulsan (KR); Seok Man KIM, Ulsan (KR)

(21) Appl. No.: 17/617,463

(22) PCT Filed: Dec. 7, 2020
(86) PCT No.: PCT/KR2020/017785

§ 371 Date: Dec. 8, 2021

(30) Foreign Application Priority Data
Nov. 25, 2020 (KR) 10-2020-0160175

(51) Int. Cl. A01K 1/00; E04D 13/04; A61L 9/14; B01D 53/18

(52) U.S. Cl. CPC A01K 1/0082; E04D 13/04; A61L 9/145; B01D 53/185; B01D 2257/90

(57) ABSTRACT

One embodiment of the present invention provides a water circulation-type malodor reduction system including a fluid supply line that supplies a fluid to a roof of a livestock barn, a first reservoir which is provided on one side of the roof and in which the fluid flowing along the roof is accommodated, and an accommodation part in which the fluid falling from the first reservoir is accommodated, wherein the accommodation part is disposed so that the fluid falls in an S shape.

### TECHNICAL FIELD

The present invention relates to a water circulation-type malodor reduction system, and more particularly, to a water circulation-type malodor reduction system in which a water curtain is formed on one side of a livestock barn, and thus malodor generated inside the livestock barn may be prevented from spreading to the surroundings.

### BACKGROUND ART

According to the recently enforced Malodor Prevention Act (Korea Ministry of Environment), regulations on the discharge of designated malodorous gases such as ammonia and hydrogen sulfide are being implemented for livestock facilities having a certain area of pig breeding facilities.

This is an act that requires that the concentrations of the designated malodorous gases measured at boundaries of sites in which the livestock facilities are located should satisfy respective discharge allowance standards, and is an act for solving the problem caused by the malodorous gases in an area adjacent to a livestock barn.

The current Malodor Prevention Act is in an early stage of implementation and is not strictly applied unlike the Wastewater Management Act. However, within the next few years, livestock farms are approaching a situation in which the livestock farms should strictly comply with the Malodor Prevention Act. Therefore, in order to satisfy the Malodor Prevention Act, the need for a system for preventing or reducing the discharge of the malodorous gas discharged from the livestock facilities is emerging.

Meanwhile, Korean Patent Application Publication No. 10-2019-0064805 (published on Jun. 11, 2019) relates to a water screen device for preventing malodor, which is installed in a box section of a river to treat malodor, and proposes a water screen device for preventing malodor, which includes a water storage unit that is installed in one upper end of a river box in a length corresponding to the width of the river, has an open upper part, has an accommodation part formed at a lower part thereof, and has the height of a front surface or rear surface of the length corresponding to the width of the river, which is lower than one surface thereof, a water supply unit that supplies water of the river to the water storage unit, and a water supply pipe that is installed at an inner lower portion of the water storage unit, is connected to the water supply unit, and supplies the water to the entire interior of the water storage unit, wherein the water stored in the water storage unit and rising overflows and falls from the one lower surface so as to form a water screen.

However, in the water screen device for preventing malodor, since the water falls at once to form a water curtain, a large number of voids are generated in a lower part of the water curtain, and thus the malodor may leak through the voids.

In addition, the water is splashed in all directions according to the falling of the water, and thus it is difficult to apply the water screen device for preventing malodor to the livestock barn.

### DISCLOSURE

### Technical Problem

The present invention is directed to providing a water circulation-type malodor reduction system capable of preventing leakage of malodor in a livestock barn by forming a uniform water curtain without voids.

### Technical Solution

One aspect of the present invention provides a water circulation-type malodor reduction system including a fluid supply line that supplies a fluid to a roof of a livestock barn, a first reservoir which is provided on one side of the roof and in which the fluid flowing along the roof is accommodated, and an accommodation part in which the fluid falling from the first reservoir is accommodated, wherein the accommodation part is disposed so that the fluid falls in an S shape.

The accommodation part may be formed in an open top shape and may be formed so that one side of the accommodation part is higher than the other side thereof.

The accommodation part may include a first accommodation part disposed below the first reservoir, and a second accommodation part disposed below the first accommodation part, and the first accommodation part and the second accommodation part may be arranged to face opposite directions to each other.

A water wheel rotatable in place may be disposed above the accommodation part, and the fluid may fall onto the water wheel before being accommodated in the accommodation part.

The water circulation-type malodor reduction system may further include a second reservoir in which the fluid falling from the accommodation part is accommodated, a recirculation line that supplies the fluid accommodated in the second reservoir to the fluid supply line, and a circulation pump disposed on a path of the recirculation line.

The first reservoir may extend along the roof and include a horizontality adjustment unit that adjusts a horizontality of the first reservoir.

The horizontality adjustment unit may include a rib extending along the roof and an adjustment member fixed to a bottom surface of the first reservoir through the rib, and when a fixing unit is tightened, the first reservoir may move closer to the rib, and when the fixing unit is loosened, the first reservoir may move away from the rib.

### Advantageous Effects

According to one aspect of the present invention, a fluid is supplied to a fluid supply line through a recirculation line and is re-supplied through a nozzle to thereby perform a cooling function on a roof of a livestock barn heated by solar heat again, and thus the total energy efficiency can be increased.

In addition, a plurality of accommodation parts are arranged between a first reservoir and a second reservoir to face opposite directions to each other. Thus, even while a space is efficiently utilized, a water curtain without voids can be uniformly formed, and accordingly, malodor in the livestock barn can be effectively prevented from leaking.

The effects of the present invention are not limited to the above effects and should be understood to include all effects that may be deduced from the detailed description of the present invention or the configuration of the present invention described in the appended claims.

### DESCRIPTION OF DRAWINGS

FIG. 1 is a perspective view of a water circulation-type malodor reduction system according to an embodiment of the present invention.

FIG. 2 is an enlarged view of the water circulation-type malodor reduction system according to the embodiment of the present invention.

FIG. 3 is a side view of the water circulation-type malodor reduction system according to the embodiment of the present invention.

FIG. 4 is an enlarged perspective view of a first reservoir according to the embodiment of the present invention.

FIG. 5 is an exploded perspective view of FIG. 4.

FIG. 6 is a schematic view illustrating a process of adjusting horizontality of the first reservoir according to the embodiment of the present invention.

### MODES OF THE INVENTION

Hereinafter, the present invention will be described with reference to the accompanying drawings. However, the present invention may be implemented in various different forms and thus is not limited to embodiments described herein. Further, in the drawings, parts irrelevant to the description are omitted in order to clearly describe the present invention, and throughout the specification, the similar numerals reference numerals are assigned to the similar parts.

Throughout the specification, when a first part is connected to a second part, this includes not only a case in which the first part is “directly connected” to the second part but also a case in which the first part is “indirectly connected” to the second part with a third part interposed therebetween. Further, when a part “includes” a component, this means that another component is not excluded but may be further included unless otherwise stated.

The terms including an ordinal number such as “first” or “second” used herein may be used to describe various components or steps, but the components or steps should not be limited by the ordinal number. The terms including an ordinal number should be construed only for distinguishing one component or step from other components or steps.

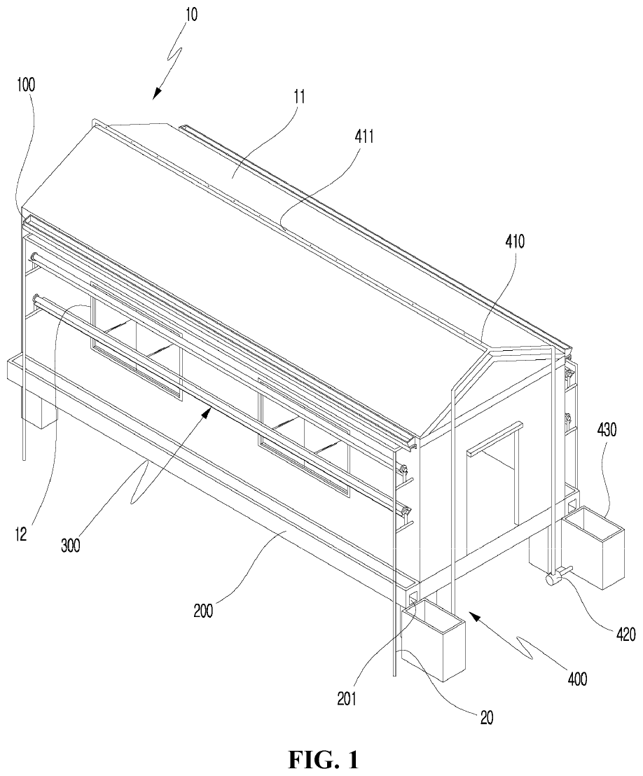

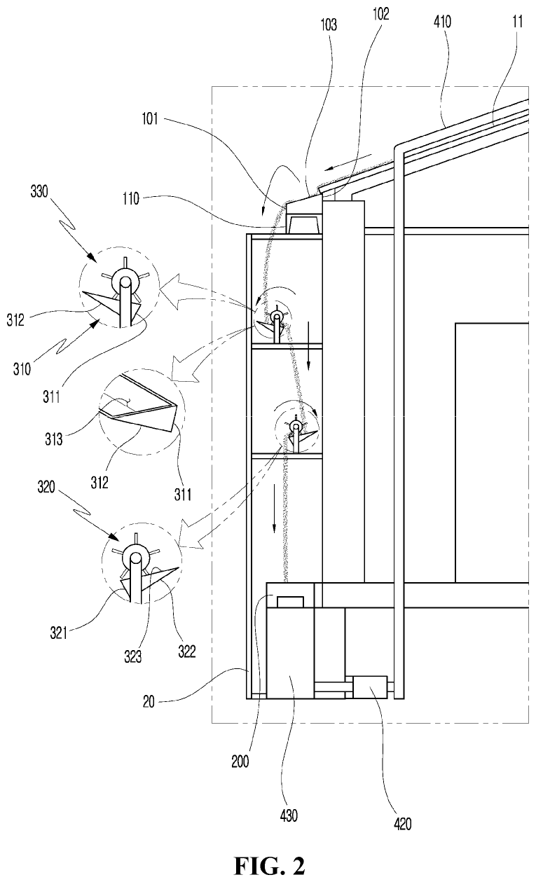

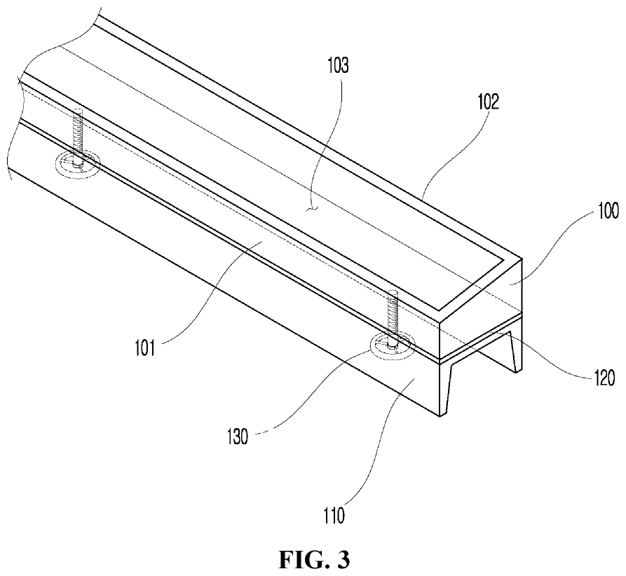

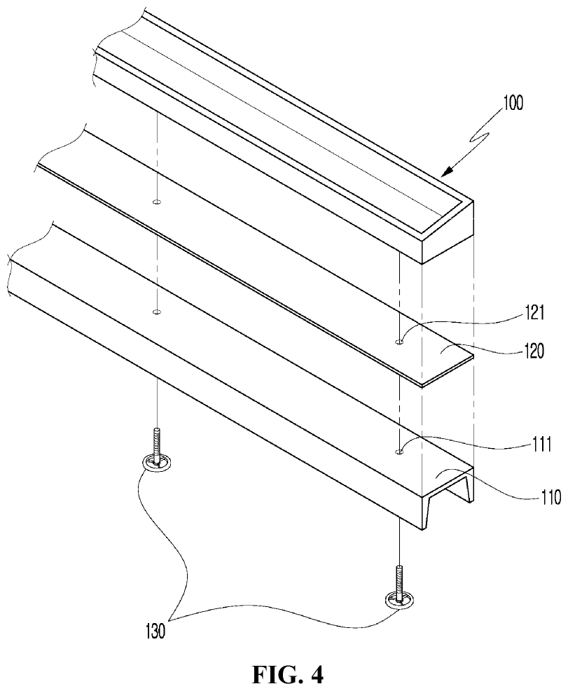

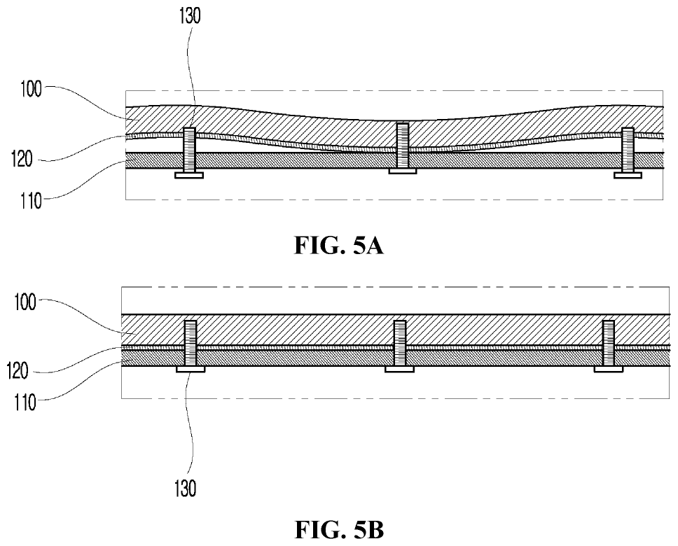

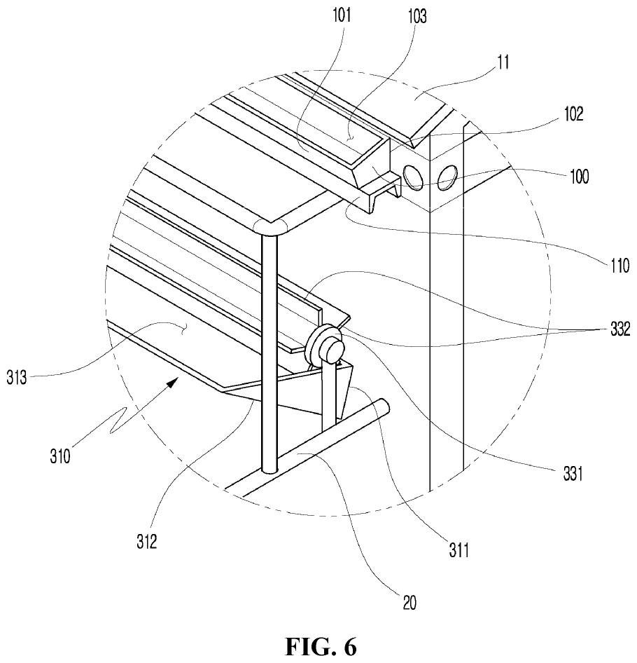

FIG. 1 is a perspective view of a water circulation-type malodor reduction system according to an embodiment of the present invention. FIG. 2 is an enlarged view of the water circulation-type malodor reduction system according to the embodiment of the present invention. FIG. 3 is a side view of the water circulation-type malodor reduction system according to the embodiment of the present invention. FIG. 4 is an enlarged perspective view of a first reservoir according to the embodiment of the present invention. FIG. 5 is an exploded perspective view of FIG. 4. FIG. 6 is a schematic view illustrating a process of adjusting horizontality of the first reservoir according to the embodiment of the present invention.

Hereinafter, embodiments of the present invention will be described in detail with reference to the accompanying drawings.

As illustrated in FIG. 1, a roof 11 may be formed on a ceiling of a livestock barn 10, and a plurality of open windows 12 may be formed in side walls of the livestock barn 10. The roof 11 may mean a roof 11 that is generally installed, has an isosceles triangular shape, and has a highest edge of a length corresponding to the ceiling of the livestock barn 10, and the open windows 12 may mean a passage through which malodor generated from excrement or the like of livestock inside the livestock barn 10 is discharged to the outside.

A nozzle 411 may be disposed at the highest edge of the roof 11. The nozzle 411 may be installed in a middle of a fluid supply line 410 included in a recirculation line 400 and sprays a fluid from an outer circumferential surface of the fluid supply line 410 in an inclined direction of the root 11. Preferably, the plurality of nozzles 411 spaced apart from each other at regular intervals may be formed over the entire length of the fluid supply line 410, and thus the entire roof 11 may be cooled.

More preferably, the nozzles 411 may be arranged on both sides of the fluid supply line 410 to spray a fluid toward both sides of the roof 11.

That is, when the roof 11 is heated by solar heat and when high-temperature heat of the roof 11 is transferred into the livestock barn 10, there is a risk that the livestock inside the livestock barn 10 may die. However, as described above, the fluid sprayed by the nozzles 411 may perform a cooling function while flowing down along the roof 11.

Here, the fluid may mean water supplied from a water supply, and in addition, the fluid may be a working fluid in which a deodorant is included in the water to provide a cleaning function.

The nozzles 411 may be formed in a shape of a hollow pipe having a predetermined length and may have a smaller diameter than the diameter of the fluid supply line 410 so that the fluid may be injected at a high pressure. Here, the fluid supply line 410 is formed at a highest point of the roof 11 and is formed to be parallel to eaves.

The fluid sprayed from the nozzles 411 falls along the roof 11 but may be accommodated in a first reservoir 100 formed below the eaves of the roof 11.

As illustrated in FIGS. 2 and 3, the first reservoir 100 may be formed to have a length corresponding to the eaves and may be provided with an accommodation groove 103 in which a predetermined amount of the fluid flowing along the roof 11 due to a weight thereof may be stored.

Here, a front plate 101 of the first reservoir 100 may be formed to have a lower height than a rear plate 102, and an upper end of a side plate may be inclined to be connected from the rear plate 102 to the front plate 101.

Accordingly, when the amount of the fluid accumulated in the first reservoir 100 reaches the height of the front plate 101 or higher, the fluid falls over an upper end of the front plate 101 and thus is accommodated in a second reservoir 200 located on the ground, and when the fluid falls, a water curtain is formed.

The second reservoir 200 may be also formed to have a length corresponding to the eaves like the first reservoir 100, may have an accommodation space in which a predetermined amount of the fluid may be stored, and may be provided with an opening 201 in one side thereof so that the fluid flows to a water storage vessel 430.

Meanwhile, as the fluid falls, the potential energy of the fluid is gradually converted into the kinetic energy, and thus the flow becomes non-uniform. Therefore, when the fluid falls directly from the roof 11 of the livestock barn 10 to the second reservoir 200, a large number of voids are formed in a surface of the water curtain. Thus, the malodor in the livestock barn 10 may leak through the voids, and the water is splashed in all directions during the falling process.

In order to solve this problem, in the water circulation-type malodor reduction system according to the embodiment of the present invention, an accommodation part 300 in which the fluid may be temporarily accommodated is formed between the first reservoir 100 and the second reservoir 200.

In detail, the accommodation part 300 according to the embodiment of the present invention includes a first accommodation part 310. As illustrated in FIGS. 1 to 3, the first accommodation part 310 is located below the first reservoir 100 and has a shape having an open upper part. In addition, the first accommodation part 310 may be formed to have a length corresponding to the eaves like the first reservoir 100 and may be provided with an accommodation groove 313 in which a predetermined amount of the fluid flowing due to a weight thereof may be stored.

A one side front plate 311 of the first accommodation part 310 may be formed to have a lower height than the other rear plate 312, and an upper end of a side plate may be inclined to be connected from the rear plate 312 to the front plate 311. The first accommodation part 310 may be supported by an outer frame 20 formed in a grid shape on a lateral side of the livestock barn 10.

In this case, the first accommodation part 310 may have a triangular shape defined by the front plate 311, the rear plate 312, and the side plate, but the present invention is not limited thereto, and various shapes may be applied as long las the front plate 311 is formed to have a lower height than the rear plate 312.

The fluid falling from the first reservoir 100 is accommodated in the first accommodation part 310, and when the amount of the fluid reaches the height of the front plate 311 of the first accommodation part 310 or higher, the fluid falls over an upper end of the front plate 311. In this case, when the fluid falling from the first accommodation part 310 falls in a direction away from the livestock barn 10, the second reservoir 200 located on the ground should be spaced apart from the livestock barn 10 in response to the falling fluid, which is inefficient in terms of space utilization.

Thus, the first accommodation part 310 may be disposed in a direction opposite to the first reservoir 100 and divert the flow of the fluid in the opposite direction. To this end, the first accommodation part 310 may be disposed so that the front plate 311 faces the livestock barn 10 and may be disposed so that the rear plate 312 faces a direction away from the livestock barn 10. In this case, when the amount of the fluid reaches the height of the front plate 311 of the first accommodation part 310 or more, the fluid falls toward the livestock barn 10.

That is, the flow of the fluid is changed from the first reservoir 100 via the first accommodation part 310 so as to form a water curtain having an S shape. In addition, since a falling distance of the fluid is shortened, the fluid is accommodated in the accommodation groove 313 of the first accommodation part 310 before the flow of the fluid becomes non-uniform, thereby reducing water splashing and forming a uniform water curtain without voids. Moreover, since the second reservoir 200 may be disposed adjacent to the livestock barn 10, a space can be efficiently utilized.

Meanwhile, the area of the water curtain is formed so that a projection area of the side wall of the livestock barn 10 may cover all of the plurality of open windows 12. That is, the water curtain may function to simultaneously block and filter the malodor discharged to the outside through the open windows 12.

More preferably, the accommodation part 300 may be provided as a plurality of accommodation parts. In detail, as illustrated, the first accommodation part 310 and a second accommodation part 320 may be arranged below the first reservoir 100. In this case, the fluid falling from the first accommodation part 310 is accommodated in the second accommodation part 320, and the fluid falling from the second accommodation part 320 is accommodated in the second reservoir 200.

Preferably, the first accommodation part 310 and the second accommodation part 320 may be arranged in opposite directions to form the water curtain having an S shape.

That is, in the first accommodation part 310, the front plate 311 is disposed to face the livestock barn 10, and the rear plate 312 is disposed in a direction away from the livestock barn 10. In the second accommodation part 320, a front plate 321 is disposed in a direction away from the livestock barn 10, and a rear plate 322 is disposed to face the livestock barn 10.

In this way, when the plurality of accommodation parts 300 are arranged, the falling distance of the fluid becomes shorter, and thus the water curtain may be uniformly formed.

More preferably, as illustrated in FIG. 1, the first reservoir 100, the plurality of accommodation parts 300, and the second reservoir 200 may be arranged on each of both sides of the livestock barn 10. In this case, the leakage of a malodorous gas discharged from the open windows 12 formed on both side walls of the livestock barn 10 may be prevented.

Meanwhile, the water circulation-type malodor reduction system further includes a water wheel 330 disposed above the accommodation part 300 in order to minimize the water splashing generated as the fluid coincides with the fluid already accumulated in the first accommodation part 310 or the second accommodation part 320 when the fluid falls from the first reservoir 100 to the first accommodation part 310 or falls from the first accommodation part to the second accommodation part 320.

In detail, as illustrated in FIGS. 2 and 3, the water wheel 330 includes a cylindrical central part 331 rotatably supported by the outer frame 20 and a plurality of blades arranged along an outer circumference of the central part 331, and extends in a lengthwise direction of the accommodation part 300. In addition, the water wheel 330 is supported by the outer frame 20 to be rotatable in place, and when the fluid falls in an accommodation space between the blades 332 and more than a certain amount of fluid is accommodated, the water wheel 330 rotates in one direction due to a weight thereof, and the fluid accommodated in the accommodation space falls into the accommodation part 300 located therebelow according to the rotation of the water wheel 330.

In this case, the fluid does not fall into the fluid accumulated in the accommodation part 300 but falls into the accommodation space of the water wheel 330, the inside of which is continuously emptied according to the rotation of the water wheel 330, and thus water splashing can be reduced. In addition, since the water wheel 330 and the accommodation part 300 are arranged adjacent to each other, the water splashing does not occur when the water falls from the water wheel 330 to the accommodation part 300. That is, as described above, as the fluid falls into the water wheel 330, the water splashing can be effectively reduced.

Meanwhile, the fluid accommodated in the second reservoir 200 flows to the water storage vessel 430 through the opening 201 formed on one side thereof and circulates toward the fluid supply line 410 disposed in the roof 11 of the livestock barn 10.

In this case, as a circulation pump 420 is disposed in a middle of the recirculation line 400, a pressure is provided to raise the fluid accommodated in the second reservoir 200 to the fluid supply line 410 of the roof 11.

That is, the fluid falling into the second reservoir 200 passes through the circulation pump 420 via the recirculation line 400 and then reaches the nozzles 411 of the roof 11 through the fluid supply line 410.

Hereinafter, a process of forming a water curtain of the water circulation-type malodor reduction system according to the embodiment of the present invention will be described in detail.

The plurality of nozzles 411 are provided in the fluid supply line 410, which is formed at the highest point of the roof 11 and extends in a lengthwise direction of the eaves, in a lengthwise direction of the fluid supply line 410, and uniformly supply the fluid to the entire surface of the roof 11. The fluid flows downward along the roof 11 formed to be inclined and is accommodated in the accommodation groove 103 of the first reservoir 100 provided on one side of the roof 11.

Here, when the level of the fluid in the accommodation groove 103 increases and the level becomes higher than the height of the front plate 101 of the first reservoir 100, the fluid flows over the upper end of the front plate 101 and falls downward. Next, the fluid falls into the accommodation space between the blades 332 of the water wheel 330 located above the first accommodation part 310, and the fluid is accommodated in the accommodation groove 313 of the first accommodation part 310 located therebelow according to the rotation of the water wheel 330.

When the level of the fluid of the first accommodation part 310 is increased, the height of the fluid becomes higher than the height of the front plate 311 of the first accommodation part 310, and thus when the fluid flows over the upper end of the front plate 311, the fluid falls downward. In this case, the front plate 311 of the first accommodation part 310 is disposed to face the livestock barn 10 as opposed to the first reservoir 100, and thus the flow of the fluid is diverted to form the water curtain having an S shape.

The fluid falls onto the water wheel 330 located above the second accommodation part 320 and is then accommodated in the accommodation groove 323 of the second accommodation part 320. When the level of the fluid of the second accommodation part 320 becomes higher than the front plate 321 of the second accommodation part 320, the fluid flows over an upper end of the front plate 321 and falls into the second reservoir 200 located therebelow. In this case, the front plate 321 of the second accommodation part 320 is disposed to face a direction away from the livestock barn 10 as opposed to the first reservoir 100, and thus the flow of the fluid is diverted again to form the water curtain having an S shape.

Thereafter, the fluid accommodated in the second reservoir 200 passes via the water storage vessel 430 of the recirculation line 400 through the opening 201 provided on one side of the second reservoir 200, passes through the circulation pump 420, and then reaches the fluid supply line 410 again.

The fluid is supplied to the fluid supply line 410 through the recirculation line 400 and is re-supplied through the nozzles 411 to thereby perform a cooling function on the roof 11 of the livestock barn 10 heated by solar heat again, and thus the total energy efficiency can be increased.

In addition, the plurality of accommodation parts 300 are arranged between the first reservoir 100 located on one side of the roof 11 of the livestock barn 10 and the second reservoir 200 located on the ground, and the accommodation parts 300 are arranged to face directions opposite to each other. Thus, even while the space is efficiently utilized, the water curtain can be uniformly formed, and accordingly, the malodor in the livestock barn 10 can be effectively prevented from leaking.

Moreover, the fluid falls onto the water wheel 330 located above the accommodation part 300, and thus the water splashing can be reduced.

Meanwhile, when the first reservoir 100 according to the embodiment of the present invention extends long in the lengthwise direction of the eaves, the first reservoir 100 may be bent due to various environmental factors in the course of use. In this case, the water is intensively supplied to the bent portion, and thus the water curtain cannot be uniformly formed in the lengthwise direction of the eaves.

Accordingly, the first reservoir 100 of the present invention includes a horizontality adjustment unit.

In detail, as illustrated in FIGS. 4 and 5, the horizontality adjustment unit includes a rib 110 having an open lower part and a “⊂” shape and extending in the lengthwise direction of the eaves, a fixed plate 120 disposed on an upper surface of the rib 110, and an adjustment member 130 passing through the rib 110 and the fixed plate 120 to be fixed to the first reservoir 100.

The rib 110 is supported by an upper end of the outer frame 20, and a plurality of through-holes 111 and 121 are arranged in the rib 110 and the fixed plate 120 at regular intervals in a lengthwise direction thereof. In this case, fixing grooves (not illustrated) are also formed on a bottom surface of the first reservoir 100 at locations corresponding to the through-holes 111 and 121.

The through-holes 111 and 121 of the rib 110 and the fixed plate 120 and the fixing grooves of the first reservoir 100 are aligned with each other so that the centers thereof coincide with each other, and next, the adjustment member 130 passes through the through-holes 111 and 121 to be fixed to the fixing grooves. In this case, a screw thread may be formed on an outer peripheral surface of the adjustment member 130, and tapped holes corresponding to the screw thread may be formed in the through-holes 111 and 121 and the fixing grooves.

Accordingly, when the adjustment member 130 is tightened, a part of the first reservoir 100, in which the adjustment member 130 is located, is moved downward to be more firmly fixed to the rib 110, and when the adjustment member 130 is loosened, a force applied to the first reservoir 100 is reduced, and thus the part of the reservoir 100 may be moved upward.

Accordingly, as illustrated in FIG. 6, when the part of the first reservoir 100 is bent upward, when the adjustment member 130 near the bent part is firmly tightened, the bent part is moved downward and is fixed to the rib 110 so as to maintain a horizontal state.

In addition, when the part of the first reservoir 100 is bent downward, when the adjustment member 130 near the bent part is loosened, the bent part is moved upward to maintain a horizontal state again.

That is, when the first reservoir 100 is bent, the adjustment members 130 arranged at regular intervals in a lengthwise direction may be tightened or loosened without having to horizontalize the first reservoir 100 after the entire first reservoir 100 is replaced or separated, thereby maintaining the horizontality of the first reservoir 100, and thus management is easy.

Meanwhile, the water circulation-type malodor reduction system according to the embodiment of the present invention may further include a control unit (not illustrated) that controls an output amount of the circulation pump 420 and a malodor detection sensor (not illustrated) that detects the malodor to provide a signal to the control unit.

In detail, the malodor detection sensor may measure the intensity of the malodor leaking through the open windows 12 of the livestock barn 10 and provide the signal to the control unit, and when the measured intensity of the malodor is higher than a preset value, the control unit may increase the output amount of the circulation pump 420 to increase the amount of fluid sprayed from the roof 11. In this case, the amount of fluid falling from the roof 11 of the livestock barn 10 is increased, the thickness of the water curtain is increased, and thus the leakage of the malodor can be further blocked.

In this case, a sensor, which is a total reduced sulfur (TRS) sensor, for total reduced sulfur components such as hydrogen sulfide, methyl mercaptan, methyl sulfide, and methyl disulfide, which have high odor intensity and odor contribution, is used as the malodor detection sensor.

The above description of the present invention is merely illustrative, and those skilled in the art to which the present invention pertains can understand that the present invention can be easily modified in other specific forms without changing the technical spirit or essential features of the present invention. Therefore, it should be understood that the embodiments described above are illustrative but not limiting in all aspects. For example, components described as a single type may be implemented in a distributed manner, and likewise, components described in a distributed manner may also be implemented in a coupled form.

The scope of the present invention is indicated by the appended claims, and all changes or modifications derived from the meaning and scope of the appended claims and equivalent concepts thereof should be construed as being included in the scope of the present invention.

1. A water circulation-type malodor reduction system comprising:
a fluid supply line that supplies a fluid to a roof of a livestock barn;
a first reservoir which is provided on one side of the roof and in which the fluid flowing along the roof is accommodated; and
an accommodation part in which the fluid falling from the first reservoir is accommodated,
wherein the accommodation part is disposed so that the fluid falls in an S shape.

2. The water circulation-type malodor reduction system of claim 1, wherein the accommodation part is formed in an open top shape and is formed so that one side of the accommodation part is higher than the other side thereof.

3. The water circulation-type malodor reduction system of claim 2, wherein the accommodation part includes:
a first accommodation part disposed below the first reservoir; and
a second accommodation part disposed below the first accommodation part, and
the first accommodation part and the second accommodation part are arranged to face opposite directions to each other.

4. The water circulation-type malodor reduction system of claim 2, wherein a water wheel rotatable in place is disposed above the accommodation part, and the fluid falls onto the water wheel before being accommodated in the accommodation part.

5. The water circulation-type malodor reduction system of claim 1, further comprising:
a second reservoir in which the fluid falling from the accommodation part is accommodated;
a recirculation line that supplies the fluid accommodated in the second reservoir to the fluid supply line; and
a circulation pump disposed on a path of the recirculation line.

6. The water circulation-type malodor reduction system of claim 1, wherein the first reservoir extends along the roof and includes a horizontality adjustment unit that adjusts a horizontality of the first reservoir.

7. The water circulation-type malodor reduction system of claim 6, wherein the horizontality adjustment unit includes:
a rib extending along the roof; and
an adjustment member fixed to a bottom surface of the first reservoir through the rib, and
when a fixing unit is tightened, the first reservoir moves closer to the rib, and when the fixing unit is loosened, the first reservoir moves away from the rib.
